(19) United States
(12) Patent Application Publication
(10) Pub. No.: US 2002/0005073 A1
(43) Pub. Date: Jan. 17, 2002

(54) Method and apparatus for testing the strength of autologous tissue

(76) Inventors: David Tompkins, Walton-on-Thames (GB); David Hemsley, Rickmansworth (GB)

Correspondence Address: KNOBBE MARTENS OLSON & BEAR LLP, 620 NEWPORT CENTER DRIVE, SIXTEENTH FLOOR, NEWPORT BEACH, CA, 92660, US

(21) Appl. No.: 09/838,738

(22) Filed: Apr. 19, 2001

Related U.S. Application Data
(60) Provisional application No. 60/198,650, filed on Apr. 20, 2000.

(51) Int. Cl. G01N 3/08; A61F 2/24; A61F 9/00

(52) U.S. Cl. 73/835; 623/2.13; 623/912

(57) ABSTRACT

A method and an apparatus for testing the strength of autologous tissue for use in tissue leaflets for constructing artificial heart valves. A strip of tissue is cut adjacent to the edge of the tissue leaflet which is subject to the greatest stress when the tissue leaflet is mounted in the artificial heart valve. The strip of tissue is subjected to a known load produced by a spring to give a go/no go test of tissue strength. The spring is mounted in a generally X-shaped device made up of a generally linear piece having a handle at one end and two generally V-shaped pieces. The centers of all three pieces are joined to one another by a pivot. A spring is attached to the ends of the two generally V-shaped pieces which are closest to each other. The strip of tissue is mounted on the end of the generally linear shaped piece which does not have a handle and the end of the generally V-shaped pieces which is closest to the end of the generally linear piece which is attached to the strip of tissue. The end of the generally V-shaped device closest to the handle of the generally linear piece is placed in contact with the handle of the generally linear piece, stretching the spring. The spring pulls apart the ends of the X-shaped device, exerting the known load on the test strip of tissue. If the test strip breaks under the known load, the autologous tissue is not suitable for use in the artificial heart valve.

### PRIORITY CLAIM

This application claims the benefit under 35 U.S.C. §119(e) of provisional application No. 60/198,650, filed Apr. 20, 2000.

### FIELD OF THE INVENTION

This invention relates to improvements in constructing heart valves using autologous tissue.

### BACKGROUND OF THE INVENTION

Several types of heart valves are presently available for use in replacing diseased or malfunctioning heart valves in humans.

One form of heart valve is constructed from animal tissue, typically from bovine or porcine aortic valve tissue. These valves must typically be constructed in a laboratory well in advance of when they will be needed and then stored in an aldehyde solution. Skilled technicians are required to assemble these valves. The valves constructed from animal tissue have relatively short lifetimes. The short lifetimes are caused by two factors. First, there is an antigenic reaction by the body to the animal tissue which causes the tissue to calcify, making it inflexible and more susceptible to failure with time. Second, the tissue is often stored in glutaraldehyde before implantation to try to decrease the antigenic reaction. The aldehyde tends to tan the tissue to a leather-like consistency. The repeated stress of opening and closing tends to cause the tissue to wear out.

Mechanical heart valves are also available. These valves are made from hard, non-biological materials such as metals or ceramics. Although the mechanical heart valves are durable, the hard, non-biological surfaces on the valves tend to cause blood clots. The blood clots can cause heart attacks or strokes, and, as a result, patients with mechanical heart valves must take anticoagulant drugs. These drugs can lead to hemorrhagic complications. Also, patients who take these drugs require frequent and life-long laboratory tests of their clotting time.

Another type of heart valve, the autologous tissue valve, is constructed with the patient's own tissue. A number of patents for autologous tissue heart valves and methods of making autologous tissue heart valves have issued to Autogenics, assignee of this application, including U.S. Pat. Nos. 5,161,955 and 5,326,371 and pending U.S. application Ser. No. 09/161,809, hereby incorporated herein by reference.

### SUMMARY OF THE INVENTION

One aspect of the invention provides an improved method for constructing an autologous heart valve. During construction of this type of valve, the individual or individuals building the valve currently rely upon their judgement and experience as to whether the harvested tissue is of adequate quality to allow a durable valve to be built. Because these valves are built during open heart surgery, there is only a limited amount of time for testing the mechanical properties of the available tissue.

As described below, the embodiments of the invention provide a simple go/no go test of tissue strength using a strip of tissue cut from the valve material adjacent to the edge of the tissue leaflet subject to the greatest stress when the tissue leaflet is mounted in the artificial heart valve.

### BRIEF DESCRIPTION OF THE DRAWINGS

FIG. 1 is a perspective view of a preferred embodiment of an assembled autologous heart valve;

FIG. 2A is a front view of an autologous tissue leaflet cut so as to include a test strip portion;

FIG. 2B is a front view of the autologous tissue leaflet of FIG. 2A, after the test strip portion is cut off for testing; and

FIG. 3 is a plan view of a tissue loading device constructed in accordance with an embodiment of the invention.

### DETAILED DESCRIPTION OF THE PREFERRED EMBODIMENT

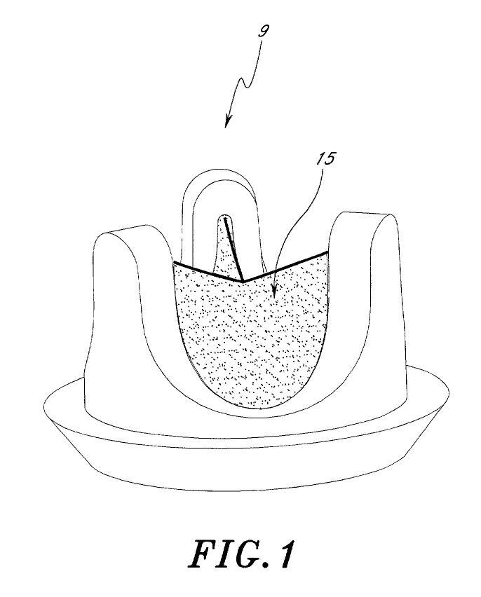

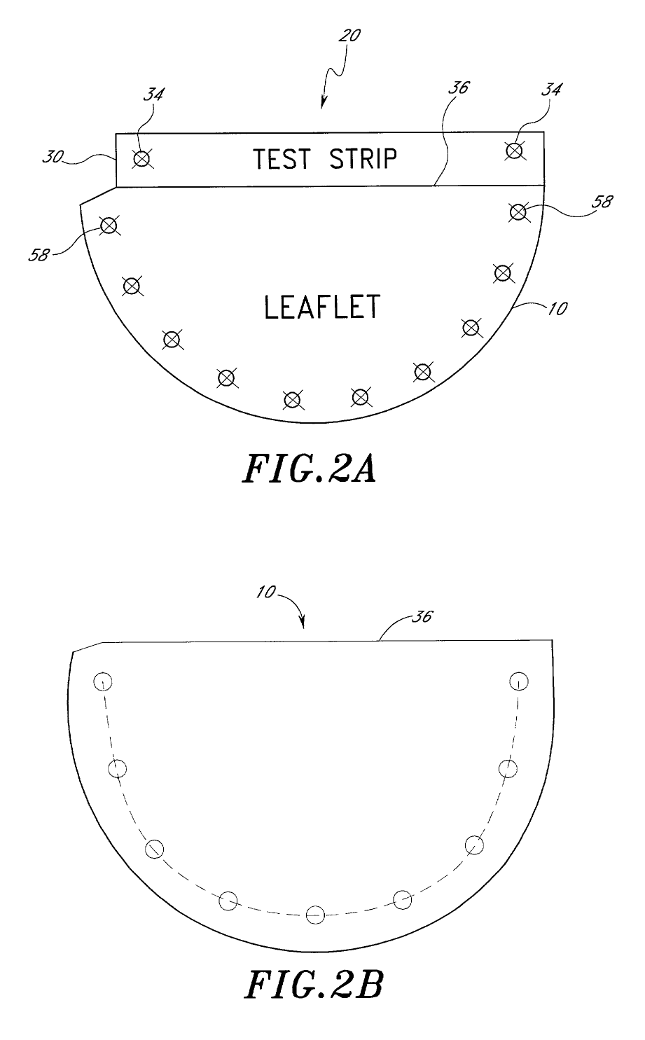

FIG. 1 illustrates an exemplary embodiment of an assembled heart valve 9. This valve uses the patient's own tissue and is constructed intraoperatively from several factory manufactured components. These components include a tissue mounting frame that mounts three individual autologous tissue leaflets 10, one such leaflet being shown in FIG. 2B. The final assembled configuration of the three leaflets is shown at 15 in FIG. 1. This type of valve is designed to be intraoperatively assembled by the surgeon during an open heart procedure. Typical assembly times are of the order of 10 minutes.

During construction of the valve, the individual or individuals building the valve currently rely upon their judgement and experience as to whether the harvested tissue is of adequate quality to allow a durable valve to be built. Because the valves are built during open heart surgery, there is only a limited amount of time for testing the mechanical properties of the available tissue. Among the many factors influencing the strength of the autologous tissue are the collagen quality, its cross linking (the effect of glutaraldehyde treatment), the direction of the main mass of collagen fibers, and the proportion of collagen within the tissue mass. The methods which are currently available to determine these parameters are time consuming.

As described below, an embodiment of the method provides a simple go/no go test of tissue strength using a strip of tissue cut from the valve material adjacent to the edge of the tissue leaflet subject to the greatest stress when the tissue leaflet is mounted in the artificial heart valve.

Early Finite Element Analysis (FEA) work on the stresses within a leaflet indicates that the highest stress points occur at points close to the coaptation line near the anchor positions at the top of the tissue leaflet. These highest stresses can be related to what is known as the “membrane” stress. The membrane stress can be visualized as the stress that is found in the rubber of an inflated balloon. The highest stress in the tissue leaflet is always higher than the membrane stress, and the quality of the design is related to how close the maximum stress in the tissue leaflet is to the membrane stress.

However, in the balloon and the valve leaflet, the important strength factor is not the ultimate allowable stress but the ultimate allowable load per unit width of the material. Thus, if a constant width of tissue is tested, there will be a minimum load which the strip of tissue must be able to withstand. This minimum load is independent of the tissue thickness. Thus, a thick tissue with a low ultimate tensile stress can be matched in strength by a thin tissue with a high ultimate tensile stress.

If the test sample is taken from a position close to the leaflet's highest stress point, the differences due to possible collagen alignment are minimized. The close coupling of the sample to the portion of the leaflet subject to the highest stress also minimizes the effects of any other limiting factors such as inadequate fixing.

FIG. 2A shows a test tissue leaflet 20 cut to include a test strip portion 30 adjacent the portion of the tissue leaflet subject to the highest amount of stress, the top anchor points 58. The test tissue leaflet 20 of FIG. 2A includes the test strip portion 30 and the tissue leaflet 10 of FIG. 2B. In an exemplary embodiment, the test strip portion 30 includes a test strip tissue hole 34 near each end of the test strip portion 30. In a series of laboratory tests to evaluate pericardium, a standard strip width of 5 mm has been generally accepted as a reasonable compromise between minimizing the amount of tissue and what would provide an ideal test sample shape. In an exemplary embodiment, the test strip portion 30 is therefore selected to be approximately 5 mm wide.

Tissue leaflets are typically cut with a tissue cutting die. Examples of cutting dies suitable for cutting predetermined shapes in autologous tissue are shown and described in U.S. Pat. Nos. 5,163,955 and 5,425,741, hereby incorporated herein by reference. In order to produce a test tissue leaflet 20 with the shape shown in FIG. 2A with the test strip portion 30, the cutting die can be modified to provide the test strip portion 30 with the desired qualities.

Another form of cutting die which is suitable for cutting tissue leaflets is the rotatable tissue die shown and described in U.S. Pat. No. 5,609,600, hereby incorporated herein by reference. Although the shape of the tissue leaflet produced by the cutting die of U.S. Pat. Nos. 5,163,955; 5,425,741; and 5,609,600 is different than the shape of the test tissue leaflet 20 shown in FIG. 2A, the cutting dies in the above-referenced patents can be modified to produce the test tissue leaflet 20 with the test strip portion 30 as shown in FIG. 2A.

In an exemplary embodiment, the location of the test strip portion 30 is selected so that it is close to the portion of the tissue leaflet 10 subject to the highest load, the top anchor points 58. This location is also the best compromise for the alignment with the direction of these loads. In an embodiment, a coaptation line 36 can be used as one of the sides of the test strip portion 30 in order to minimize the tissue usage.

FIG. 2B shows the autologous test tissue leaflet 20 of FIG. 2A after the test strip portion 30 has been cut off for testing. The test strip portion 30 can be cut off from the test tissue leaflet 20 by any suitable means, for example, with a scalpel, a cutting die, or a laser cutting device. After the test strip portion 30 has been evaluated to assess the strength of the tissue, the remaining autologous tissue leaflet 10 as shown in FIG. 2B can be used in the fabrication of an assembled autologous heart valve 9 such as shown in FIG. 1.

In an exemplary alternative embodiment, the cutting die produces the autologous tissue leaflet 10 and the test strip portion 30 in a single operation without the need to separate the test strip portion 30 from the test tissue leaflet 20 in a second cutting step. In an embodiment, the coaptation line 36 is used as one of the sides of the test strip portion 30 when the tissue leaflet 10 and the separate test strip portion 30 are simultaneously produced in a single operation.

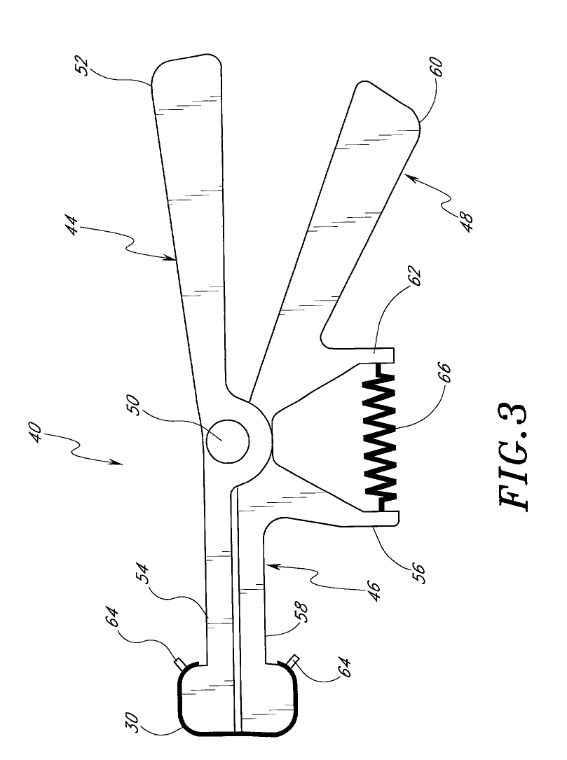

FIG. 3 shows an embodiment of a tissue testing device 40 which is suitable for testing the strength of the test strip portion 30 of the test tissue leaflet 20 of FIG. 2A. The tissue testing device 40 of FIG. 3 has a generally X-shaped frame having a generally linear first piece 44, a generally V-shaped second piece 46, and a generally V-shaped third piece 48. The generally linear first piece 44, the generally V-shaped second piece 46, and the generally V-shaped third piece 48 are pivotally joined to one another by a pivot 50 at or near the center of the generally linear first piece 44, the generally V-shaped second piece 46, and the generally V-shaped third piece 48.

The generally linear first piece 44 has a top handle 52 and an upper arm 54. The generally V-shaped second piece 46 includes a second piece loading lever 56 and a lower arm 58. The third piece has an actuating handle 60 and a third piece loading lever 62.

In an exemplary embodiment, the ends of both the upper arm 54 and the lower arm 58 are curved. In another exemplary embodiment, a projection 64 is attached to each of the curved ends. The second piece loading lever 56 and the third piece loading lever 62 are joined by a load spring 66 having a known load.

The test strip portion 30 of the test tissue leaflet 20 may be tested for strength as follows. The test strip portion 30 is attached to the tissue testing device 40 by placing the test strip tissue holes 34 over the projections 64 on the upper arm 54 and the lower arm 58 so that the length of the test strip portion 30 extends over the ends of the upper arm 54 and the lower arm 58. The top handle 52 of the generally linear first piece 44 and the actuating handle 60 of the generally V-shaped third piece 48 are squeezed together to place the top handle 52 in contact with the actuating handle 60. The load spring 66 pulls the first piece loading lever 56 and the second piece loading lever 62 toward one another, exerting the known load of the load spring 66 on the test strip portion 30 by pulling apart the ends of the upper arm 54 and the lower arm 58.

If the test strip portion 30 breaks under the known load of the load spring 66, the tensile strength of the autologous tissue forming the test tissue leaflet 20 is considered to be unsuitable for forming a tissue leaflet 10 for use in the autologous tissue valve 9 of FIG. 1. A different portion of tissue is then chosen for forming a new test tissue leaflet 20. If the test strip portion 30 does not break under the known load of the load spring 66, the tensile strength of the autologous tissue forming the test tissue leaflet 20 is judged to be suitable, and the tissue leaflet 10 remaining after the test strip portion 30 is removed from the test tissue leaflet 20 can be used in the preparation of an autologous tissue valve such as shown in FIG. 1.

The tissue testing device 40 is therefore a suitable device for testing the tensile strength of autologous tissue to determine the suitability of the autologous tissue for use in a tissue leaflet to be mounted in an artificial heart valve.

Although described in the context of testing autologous tissue, the embodiments of the method and the apparatus may be used in testing autogenous tissue, porcine tissue, or bovine tissue. The tissue may be fixed, partially fixed, or nonfixed.

Because the load which is applied to the test strip portion 30 is a specific load which depends on the strength of the load spring 66, the determination of the strength of the load spring 66 is an important parameter. The determination of the appropriate strength of the load spring 66 is determined by one of ordinary skill in the art. The strength of the load spring 66 may be reduced as the size of the heart valve and the tissue leaflets 10 is reduced.

The tissue testing device 40 shown in FIG. 3 is to be considered only as illustrative of a suitable apparatus and method. The tissue testing device 40 may be modified in various manners. For example, an elastic rubber strip could be substituted for the load spring 66 to exert the known load on the test strip portion 30.

Although the top handle 52 and the actuating handle 60 provide a convenient way to allow the force of the load spring 66 to be exerted on the test strip portion 30, in an alternative embodiment, the handles may be omitted from the tissue testing device 40. Other forms of a suitable tissue testing device may include two linear pieces joined at the center by a pivot, similar to a pair of scissors. If a test strip portion 30 is attached to two of the adjacent ends of the scissors-like device and load spring is attached to the side of the scissors-like device at or near the ends of the device, the load spring would exert the known force on the test strip portion 30. If the test strip portion 30 does not break when subjected to the known load, the tissue is considered suitable for use in preparing a heart valve.

Various modifications and alterations of this invention will be apparent to one skilled in the art without departing from the scope and spirit of this invention. It should be understood that the invention is not limited to the embodiments disclosed therein, and that the claims should be interpreted as broadly as the prior art allows.

What is claimed is:

1. A method for testing the tensile strength of autologous tissue to determine the suitability of the autologous tissue for use in a tissue leaflet to be mounted in an artificial heart valve, said method comprising:
cutting a tissue leaflet and a test strip of autologous tissue, where the test strip is cut proximate to an edge of said tissue leaflet subject to the greatest stress when said tissue leaflet is mounted in the artificial heart valve;
applying a known load along the length of said test strip of autologous tissue, wherein said known load is greater than a load applied to said edge of said tissue leaflet subject to the greatest stress when said tissue leaflet is mounted in the artificial heart valve; and
determining whether said test strip of autologous tissue breaks when subjected to said known load, thereby determining the suitability of the autologous tissue for use as a tissue leaflet to be mounted in an artificial heart valve.

2. A method for testing the tensile strength of autologous tissue to determine the suitability of the autologous tissue for use in a tissue leaflet to be mounted in an artificial heart valve, said method comprising:
cutting a test strip of autologous tissue proximate to an edge of said tissue leaflet subject to the greatest stress when said tissue leaflet is mounted in the artificial heart valve; and
applying a known load along the length of said test strip of autologous tissue to determine if said known load causes said test strip to break.

3. A method for testing the tensile strength of autologous tissue to determine the suitability of the autologous tissue for use in a tissue leaflet to be mounted in an artificial heart valve, said method comprising:
cutting a test strip of autologous tissue proximate to an edge of said tissue leaflet subject to the greatest stress when said tissue leaflet is mounted in the artificial heart valve;
applying a known load along the length of said test strip of autologous tissue, wherein said known load is greater than a load applied to said edge of said tissue leaflet subject to the greatest stress when said tissue leaflet is mounted in the artificial heart valve; and
determining whether said test strip of autologous tissue breaks when subjected to said known load, thereby determining the suitability of the autologous tissue for use as a tissue leaflet to be mounted in an artificial heart valve.

4. A method for testing the tensile strength of autologous tissue to determine the suitability of the autologous tissue for use in a tissue leaflet to be mounted in an artificial heart valve, said method comprising:
cutting a test strip of autologous tissue proximate to an edge of said tissue leaflet subject to the greatest stress when said tissue leaflet is mounted in the artificial heart valve; and
applying a known load along the length of said test strip of autologous tissue.

5. The method of claim 4, wherein said known load is greater than a load applied to said edge of said tissue leaflet subject to the greatest stress when said tissue leaflet is mounted in the artificial heart valve.

6. The method of claim 4, including determining whether said test strip of autologous tissue breaks when subjected to said known load, thereby determining the suitability of the autologous tissue for use as a tissue leaflet to be mounted in an artificial heart valve.

7. The method of claim 4, wherein breakage of said test strip when said known load is applied is indicative that the autologous tissue is not suitable for use as a heart valve leaflet.

8. The method of claim 4, wherein the testing is performed in an operating room during open heart surgery.

9. The method of claim 4, wherein said known load is produced by a spring.

10. The method of claim 9, wherein said spring is mounted in a generally X-shaped device comprising:
a generally linear first piece;
a generally V-shaped second piece; and
a generally V-shaped third piece, wherein said generally X-shaped device has a first end comprising a first end of said generally linear first piece and a first end of said generally V-shaped second piece and a second end comprising a second end of said generally linear first piece and a first end of said generally V-shaped third piece and wherein said generally linear first piece, said generally V-shaped second piece, and said generally V-shaped third piece are pivotally joined to each other at or near a center of said generally linear first piece, said generally V-shaped second piece, and said generally V-shaped third piece, wherein said spring is attached to a second end of said generally V-shaped second piece and a second end of said generally V-shaped third piece, whereby said spring exerts said known load to open said first end of said generally X-shaped frame when said second end of said generally linear first piece and said first end of said generally V-shaped third piece are placed in contact with one another.

11. The method of claim 10, wherein a first end of said test strip of autologous tissue is attached to said first end of said generally linear first piece and a second end of said test strip of autologous tissue is attached to said first end of said generally V-shaped second piece, thereby applying said known load along the length of said test strip.

12. The method of claim 10, wherein said first end of said generally linear first piece and said first end of said generally V-shaped second piece are curved.

13. The method of claim 11, wherein said first end of said generally linear first piece and said first end of said generally V-shaped second piece each comprise a projection holding said first end and said second end of said test strip of autologous tissue.

14. The method of claim 10, wherein said device is reusable.

15. The method of claim 10, wherein said device is disposable.

16. An apparatus for testing the tensile strength of autologous tissue to determine the suitability of the autologous tissue for use in a tissue leaflet to be mounted in an artificial heart valve, said apparatus comprising:
a generally X-shaped device comprising:
a generally linear first piece;
a generally V-shaped second piece; and
a generally V-shaped third piece, wherein said generally X-shaped device has a first end comprising a first end of said generally linear first piece and a first end of said generally V-shaped second piece and a second end comprising a second end of said generally linear first piece and a first end of said generally V-shaped third piece and wherein said generally linear first piece, said generally V-shaped second piece, and said generally V-shaped third piece are pivotally joined to each other at or near a center of said generally linear first piece, said generally V-shaped second piece, and said generally V-shaped third piece; and
a spring, wherein said spring is attached to a second end of said generally V-shaped second piece and a second end of said generally V-shaped third piece, whereby said spring exerts said known load to open said first end of said generally X-shaped frame when said second end of said generally linear first piece and said first end of said generally V-shaped third piece are placed in contact with one another.

17. The apparatus of claim 16, wherein a first end of a test strip of autologous tissue is attached to said first end of said generally linear first piece and a second end of said test strip of autologous tissue is attached to said first end of said generally V-shaped second piece, thereby applying said known load along the length of said test strip.

18. The apparatus of claim 16, wherein said first end of said generally linear first piece and said first end of said generally V-shaped second piece are curved.

19. The apparatus of claim 16, wherein said first end of said generally linear first piece and said first end of said generally V-shaped second piece each comprise a projection holding said first end and said second end of said test strip of autologous tissue.

20. The apparatus of claim 16, wherein said apparatus is reusable.

21. The apparatus of claim 16, wherein said apparatus is disposable.
